(19) United States
(12) Patent Application Publication
(10) Pub. No.: US 2008/0319377 A1
(43) Pub. Date: Dec. 25, 2008

(54) Medical Device for Generating Transient Bubbles

(76) Inventor: James Keenan, Ottawa (CA)

Correspondence Address: PEARNE & GORDON LLP, 1801 EAST 9TH STREET, SUITE 1200, CLEVELAND, OH, 44114-3108, US

(21) Appl. No.: 11/579,463

(22) PCT Filed: May 4, 2005
(86) PCT No.: PCT/CA2005/000679

§ 371 Date: Mar. 26, 2008

Related U.S. Application Data
(60) Provisional application No. 60/567,453, filed on May 4, 2004.

(51) Int. Cl. A61M 5/14

(52) U.S. Cl. 604/24

(57) ABSTRACT

A method and medical device for generating transient bubbles is provided. Fluid is delivered under pressure through bubble generating means and thereby transient bubbles are generated. The bubbles are micro or nano sized. An ultrasonic source may be used to further vibrate the bubble generating means so as to adjust the size and size consistency of the bubbles. Infusion means may also be provided for infusing the bubbles in a patient. A therapeutic agent, in the form of or delivered in combination with, bubbles may be injected at the point of interest. An ultrasound pulse may additionally be provided to activate bubbles to enhance drug transport through tissues and across cell membranes. The device provides a means to locally generate transient bubbles, to facilitate acoustical activation of therapeutic agents, and the acoustic activation of bubbles infused within therapeutic agents, in order to enhance treatment efficacy.

### FIELD OF THE INVENTION

The invention generally pertains to medical devices for infusing therapeutic agents into patients and more particularly to medical devices for generating transient bubbles suitable for infusion into patients. Such devices can be used, for example, to inject or otherwise administer therapeutic agents in the form of, or in combination with, transient micro or nano bubbles. Acoustic activation of the bubbles, through the application of ultrasound energy, is used for improving treatment efficacy.

### BACKGROUND TO THE INVENTION

Acoustically activated drug delivery systems are typically administered to a patient and then activated by extracorporeal ultrasound sources to release the therapeutic compound. The cavitations that may occur upon activation can enhance the drug uptake in the patient's cells and hence improve the treatment efficacy.

A variety of acoustically activated drug delivery systems exist including a core drug encapsulated in microspheres, biodegradable polymer and drug solution bubbles, drug impregnated microsponges, injectable nanoparticles such as vesicles, micelles, and liposomes, and other drug carrying particles, bubbles, or spheres that permit acoustic activation of therapeutic agents.

The therapeutic agents may be chemotherapy drugs, gene therapy, and other agents. Due to the localized delivery method, a high dosage of toxic drugs may be delivered to a point of interest while minimizing negative side effects.

Acoustic activation technology shows promise for the treatment of drug resistant cancer tumors, vascular disease, and other diseases. Research has demonstrated improved cellular drug uptake associated with these systems. Further efficacy benefits maybe obtained by infusing transient gas microbubbles in combination with acoustically activated drug systems to enhance the local cavitation effects.

Presently, acoustically activated drug delivery systems are typically formulated in a pharmaceutical setting. They are designed to persist through to administration to a patient. Such products are required to maintain integrity through the shock, vibration, and temperature changes of transportation and to persist over time through to administration. Such formulations may include complex design and processing features, for example, are typically polymer or polymer and solvent based. However, these persistent systems must also dissipate or degrade in the patient once administered and activated. These polymers and any other components are required to biodegrade with minimal negative side effects.

Acoustically activated drug delivery systems may range in size from sub micron, nano scale, up to 1000 microns, with a one to ten micron size typical. Size preference depends upon the resonant frequency, or a harmonic, of the bubble or particle to be activated at a particular ultrasound frequency, and may also depend upon the desired release rates of the encapsulated drugs. Increased size uniformity would encourage effective and more complete activation.

Presently, key acoustically activated drug delivery system parameters, such as size and concentration of microbubbles, are not easily modified during administration.

Physicians would benefit from the means to alter bubble parameters during a procedure. The resulting effects, such as transducer signals indicating that the cavitation threshold has been reached or effects to the quality of the ultrasound image, could be monitored real time and bubble parameters adjusted as required. For example, the bubbles infused into the therapeutic agent could be of a high concentration in order to optimize the acoustic activation. The bubbles infused into a carrier fluid in order to improve the ultrasound image guidance could be of a lower concentration so as not to obscure the image.

The size of bubbles infused into the therapeutic agent could be altered depending whether the therapeutic intent was unstable or stable cavitation. Unstable cavitation typically involves gas bubbles in the order of 20 microns in diameter and these are destroyed by the ultrasound. Bubbles for stable cavitation, also referred to as sonoporation, are typically smaller, in the order of 5 microns in diameter. These bubbles are not destroyed by the ultrasound energy but resonate during activation in order to increase cell permeability.

The size of bubbles infused into the therapeutic agent could be altered depending upon the anatomy of the diseased tissue. Highly vascularized tissue could be infused with bubbles in the order of 200 nanometers in order to promote drug distribution through constricted blood vessels. For less vascularized tissue bubbles in the 3-7 micron range may be preferred.

Additionally, in the field of oncology, current treatment techniques may include administration to patients with individual ‘cocktails’ of chemotherapy drugs depending upon the indication, patient reaction to therapy, and disease stage. Oncologists do not currently have a means to provide patients with a flexible, acoustically activated therapy, i.e. the means to administer a variety of drugs, or combination of drugs, on short notice, with therapeutic enhancements such as improved drug uptake and reduced side effects.

Accordingly, there is a need for a medical device that would overcome these and other drawbacks.

### SUMMARY OF THE INVENTION

A method and medical device for generating transient micro or nano bubbles, and a system for acoustical activation of the bubbles, is disclosed.

The medical device comprises a fluid vessel for holding a fluid, a fluid delivery means operatively connected to the fluid vessel for applying a pressure and causing the fluid to travel a flow path, and a bubble generating means for generating transient bubbles comprised of the fluid. The bubble generating means is positioned along the flow path of the fluid with fluid passing through the bubble generating means termed herein as “bubble fluid”. The bubbles generated are micro or nano sized.

The bubble fluid may additionally be merged into a second fluid downstream of the bubble generating means along the flow path. The bubble fluid may form bubbles within the second fluid. The second fluid may be contained within a second vessel or within a conduit into which both the bubble fluid and the second fluid flows. Additional fluids are also contemplated. Alternatively, a temporary storage vessel may be provided along the flow path to receive and hold the bubble fluid. The storage vessel may be provided at a point sufficiently downstream of the flow path so as to receive and hold a co-mingled bubbled fluid and second fluid. As a further alternative, an injection means for receiving and delivering the bubble fluid into a body may also be provided. Such an injection means would be positioned along the flow path at a position sufficiently downstream of the bubble generating means. This way, bubble fluid, or bubble fluid co-mingled with the second fluid, may be introduced into a body at a desired location, for example, into a tissue mass, tumour, muscle, skin, organ, or other suitable structure, depending on the application.

Ultrasound may then be used to rupture or otherwise activate the bubbles (acoustic activation) at a point of interest.

The injection means includes a hollow needle, catheter, tube, or other surgical instrument that can be inserted within a body to a point of interest, for example, into a tissue mass, tumour, muscle, skin, organ, vein, artery, or other suitable structure, depending on the application, and is structured to permit fluid flow. Fluid from the vessel would be able to flow through the injection means for discharge into the body, or more specifically, at a point of interest. The term “injection means” is to be broadly understood as including various means for introducing a fluid into a body, including by active injection or passive permeation, or otherwise by infusion.

All or part of the device may be mounted on a handheld device or connected by conduit to the handheld device, such as a compressed, medical grade gas canister connected by conduit to the device.

The fluid may be a liquid or gas, including in the form of a solution, a suspension, a vapour, other fine particulate solids dissolved in a liquid vehicle, a combination, or the like, provided that it is flowable. This fluid may be added to a second fluid which may also be in the form of a solution, a suspension, a vapour, other fine particulate solids dissolved in a liquid vehicle, a combination, or the like, provided that it is flowable. Thus, the device may be used to generate gas bubbles within a liquid carrier, liquid bubbles within a liquid carrier, liquid bubbles within a gas carrier, or gas bubbles within a gas carrier. The device may be used with liquefied gas.

The fluid vessel is any vessel that can hold and dispense fluid. For example, the vessel may be in the form of a syringe and the fluid delivery means may be in the form of a plunger for the syringe or a pump. As another example, the fluid vessel may be in the form of a compressed gas vessel and the fluid delivery means in the form of a compressed gas force and suitable regulators. The term “container” may be understood as interchangeable with the term “vessel”.

The fluid vessel may contain a therapeutic fluid or a carrier fluid. A therapeutic fluid may be a therapeutic liquid, such as a liquid drug or drug in solution, or contain a therapeutic agent suspended, dissolved, carried, or otherwise conveyed in a suitable liquid vehicle including drug eluting microspheres suspended in a fluid and radiolabelled isotopes. A therapeutic agent may include a variety of drug compounds, or other medicinal or non-medicinal substances, minerals, vitamins, imaging-enhancement substances, radioactive substances, and the like, that can be carried in the liquid or gaseous fluid.

Transient micro or nano bubbles of a therapeutic agent may be introduced into a carrier fluid such as a sterile saline solution, other non-therapeutic fluid, other therapeutic fluid or the same fluid and injected into the patient. The therapeutic agent or agents could comprise the bubble fluid, with some biologically passive fluid such as medical grade CO₂ gas formed within the therapeutic liquid, or a component of the bubble fluid. Alternatively, a therapeutic fluid, such as a liquid drug, may be injected into the patient and then a carrier fluid, such as saline, may be infused with bubbles and injected into a patient

The carrier fluid may be of a low viscosity to promote the generation of or the stability of bubbles. The carrier fluid solution may include additives, natural or synthetic, to alter its viscosity.

Alternatively, a carrier fluid or therapeutic fluid infused with bubbles, or a combination of therapeutic fluid with a bubble-infused carrier fluid, or vice versa, may be discharged to a temporary storage vessel. The fluid infused with bubbles could then be drawn from the storage vessel using a syringe or other suitable fluid transfer means and injected into a patient using a needle, catheter, or other means.

Alternatively, a gas such as may be transformed into bubble form within a carrier fluid, a liquid or another gas.

Surfactants, stabilizers, and other additives may be combined with the therapeutic agents or carrier fluids in order to optimize the micro or nano bubble parameters such as stability and size. Additives may be in liquid, gas, or aerosol form. The quantity of an additive may be varied during the procedure by infusion of additional material.

An electro-mechanical or piezo means may be used to uniformly mix additives the therapeutic or carrier fluids.

Fluid properties vary with temperature. The fluid vessel or vessels, conduits, and device may include means to control the fluid temperature, such as refrigeration or heating sources, temperature sensors and controls, thermal insulating materials, and the like, to enhance the bubble generation. Temperature control means in combination with fluid pressure control, may enable a suitable fluid in liquid form to be transformed into micro bubbles and infused into the patient, after which body temperature and pressure may cause the bubbles to change to a gas form for acoustic activation. Generating liquid bubbles within a liquid carrier may be more preferable than generating gas bubbles within a liquid, while acoustically activating gas bubbles may be more effective than activating liquid bubbles.

The fluid delivery means causes fluid in the vessel to travel a flow path, usually along a conduit or vessel such as a syringe. The fluid delivery means may be a plunger on a standard medical syringe, a syringe pump, variable speed fluid transfer pump, peristaltic pump, or other means to pump fluids, and also contemplates pressurization in combination with regulators, for example, compressed gas with a gas regulator.

The fluid delivery means may be manually actuated, driven by mechanical means such as compression or extension springs, or other mechanical methods, by electro-mechanical means such as an electric motor, solenoid drive, or other electro-mechanical means, or by pneumatic or hydraulic means. Where the fluid is a compressed gas, the fluid delivery means may include compressed gas force and suitable regulators. A variety of means are contemplated and maybe selected depending on a variety of factors such as the manner of operation of the bubble generating means, the size of the bubbles, the relative viscosity of the bubble fluid and carrier fluid, and other factors, as will be appreciated. For example, the selection of a non-manual drive means for the bubble fluid may then be dependent upon sufficient pressure to deliver the fluid effectively through micro or nano sized orifices, and the rate of the flow at sufficiently higher speeds so as to tend to reduce the bubble size.

The bubble generating means may comprise a permeable interface through which the fluid is passed. The fluid may be transformed into bubbles itself or infused into a second fluid (carrier fluid). Alternatively, embodiments of the device may infuse bubbles directly into a patient without a carrier fluid, for example, where a liquid therapeutic fluid is passed through a permeable interface to be transformed into bubbles directly within a patient.

The permeable interface may be an array of tiny orifices or nozzles, machined, cast or otherwise fabricated within a solid member, or a permeable membrane, including a flexible permeable membrane, or an array of micro or nano tubes, or any suitable array of openings, spiracles, interstices, porous media, fluid passages, or orifices.

The bubble generating means may be comprised of multiple fluid pathways in order to enable the physician to alter key parameters such as bubble size. For example, fluid may be passed through a single permeable interface with larger orifices in order to generate larger bubbles for use as image guidance enhancement. The fluid path may then be switched using a valve, and the fluid passed through a permeable interface with smaller orifices, or through a series of permeable interfaces, or passed repeatedly through a single permeable interface, in order to generate smaller bubbles. Smaller bubbles may be used as acoustic activation caviation nuclei in order to enhance therapeutic effects.

The permeable interface may be vibrated, including vibration at ultrasonic frequencies with the use of a piezo transducer, piezo ceramic, piezo polymer or other such means.

A piezo source may be positioned to induce vibration in the fluid or fluid channels proximal to the permeable interface.

The benefit of vibrating the permeable interface, or fluid proximal to the permeable interface, is to produce smaller and more uniformly sized bubbles than would be produced by a static permeable interface. If small micron or sub micron bubbles were required, the vibration would simplify the fabrication of the permeable interface and may help to prevent occlusion.

The frequency of the vibration of the permeable interface may be varied in order to control the size of the bubbles. The flow rate of the fluid or fluids to be transformed into bubbles may be varied in order to control the size of the bubbles.

The permeable interface may be positioned internal or external to the patient's body.

The permeable interface may be configured in different ways, such as a coaxial needle or catheter, a hollow, permeable stylet positioned within a needle, a catheter or catheter guide wire mounted variant, a multiple lumen needle or catheter, or an extracorporeal variant such as a syringe mounted permeable interface that may be used with medical needles or catheters.

Different fluids may be delivered through alternate lumens of a coaxial needle or catheter. A fluid may then be delivered under pressure through a permeable interface in an interior needle or catheter wall, to be transformed into bubbles and intermixed with the carrier fluid.

The bubble parameters may be altered after generation. Bubbles within a carrier fluid may be vibrated prior to administration by an electro-mechanical or piezo means in order to reduce the size of bubbles.

An alternative means to generate bubbles is comprised of a nano scale valve connection between the bubble and carrier fluid conduits. The bubble fluid would be delivered under pressure to the valve and dispensed or delivered into the carrier fluid in nano or micro liter quantities. Alternatively, the bubble fluid could be dispensed or delivered directly into the patient without a carrier fluid.

Commercially available nano scale valves used for scientific instrumentation may dispense in 25 nanoliters increments or less. These nano scale valves are biocompatible and are typically comprised of valves, flex circuit electronics, Hall effect sensors, and a single-piece molded housing, and may be controlled using a driver board. Other nano scale bubble generation means include high speed ink jet nanoliter dispensers, flapper valves, rotary valves, check valves, Tesla valves with no moving parts, capillary bust valves used to regulate liquid flow in microchannels, microstructure specular spin valves with nano oxide layers, and other suitable micro and nano scale valves. Microchannels and valves may be fabricated using soft lithography.

An alternative means to generate bubbles is comprised of a nano or micron scale fluid conduit connection between the bubble and carrier fluid conduits. The bubble fluid would be delivered under pressure to the narrow, nano or micron scale fluid conduit connection and be delivered into the carrier fluid in nano or micro liter quantities. The nano or micron scale fluid conduit connection or the fluid proximal to it may be vibrated by a piezo source. Alternatively, the bubbles could be delivered directly into the patient without a carrier fluid.

A variety of means are available to produce nano or micron scale conduit including single-wall carbon nanotube based materials that are used in aerospace.

The fluid may be delivered through a hollow stylet coaxially positioned within a needle or catheter. The fluid passes through a permeable interface in the stylet to be infused with the carrier fluid flow in the needle or catheter.

The bubble fluid and carrier fluid may be delivered in separate needle or catheter lumens to a fluid connection within the patient where the permeable interface is used to generate bubbles within the carrier fluid.

The bubbles are expected to maintain their desired size and shape for a brief period of time before dissipating or otherwise altering form. If too high a percentage of bubbles dissipated prior to acoustic activation, the full therapeutic benefits would not be realized.

Generating the bubbles within a needle or catheter positioned within a patient, and immediately prior to infusion within the patient, may help to ensure that a sufficient quantity of transient bubbles have not dissipated prior to ultrasound activation.

The permeable interface may be external to the patient. The bubbles would be generated in a syringe and the bubbles and carrier fluid would be delivered through a needle or catheter and into the patient.

Forming the bubbles external to the patient may simplify treatment as standard, commercial medical needles or catheters could be used. A further benefit would be to permit the safe use of high fluid pressure bubble generation. With an extracorporeal bubble generating means, a fluid pressure ‘fuse’ may be connected between the high pressure source and the needle or catheter inserted into the patient. If the bubble fluid pressure ruptured the permeable interface or housing and introduced a high pressure source to the flow path leading directly to the patient, the fuse would rupture and reduce the fluid pressure to permissible levels.

The embodiment of the device to generate bubbles without a carrier fluid may consist of a needle, hollow stylet, or catheter with the permeable interface mounted at or in proximity to the distal tip. Fluid would be delivered through the needle, hollow stylet, or catheter and pass through the permeable interface to be infused directly within the patient in bubble form.

The therapeutic agent may be delivered to the point of interest prior to, simultaneously with, or after the delivery of the transient bubbles in a carrier fluid. The device may include fluid reservoirs for the therapeutic agent or agents, bubble fluid or fluids, and carrier fluid or fluids.

Needleless infusion devices, which can propel liquids or powders at high speeds through a patient's skin, could be used to deliver therapeutic agent. The bubble and carrier fluid, and additional therapeutic agents if required, could then be delivered to the same point of interest by needle or catheter means. An ultrasound source would then be used to acoustically activate the infused bubbles and the resulting local cavitation may enhance the efficacy of the therapeutic agent delivered by the needleless infusion device.

Devices of the present invention includes means for providing nano or micro bubbles through patient infusion means such as a needle or catheter, to enhance the therapeutic efficacy of a drug.

The device may be comprised of a handheld assembly or of a system, comprised of a handheld assembly connected to other components which may include fluid vessels, pumps, power sources, regulators, meters, and the like.

Using a system, method and medical device of the present invention, transient bubbles may be generated locally, including just prior to or during a drug administration procedure, and acoustically activated without substantial delay. This may result in a less complex therapy system, reduce the need of additives to preserve transient bubbles in pharmaceutical formulations and produce more uniform bubbles. These and other advantages will become apparent to the skilled person.

It is to be appreciated that reference to a “device” of the present invention may be understood to include an “apparatus” or “assembly”, which may be incorporated into systems with suitable adaptations.

It is also to be appreciated that the devices of the present invention may be used in a variety of applications, including medical diagnosis, image guided intervention, treatment, surgery, and the like, and also maybe used in a similar fashion in veterinary applications with suitable modifications.

The term “needle” is intended to include any hollow, slender instrument that may be manipulated to puncture or be inserted or otherwise probe tissues, organs, cavities, or the like. The needle may be used to introduce material into or remove material from a patient or to perform other therapeutic or diagnostic functions. The term needle is intended to include rods or wire-like medical instruments, cannulas, probes, tubes and lumens, stylets, and the like.

The term “patient” may be any suitable animal, including humans and other mammals.

The term “catheter” is intended to include any flexible surgical instrument for the introduction of fluids into the body, including catheters for repeat dose drug delivery such as hickman lines, PORTACATH™ lines and the like.

The fluids container may be any suitable vessel to contain gases or liquids, such as syringes, gas tanks, a central, building-supply, fluid source that may be connected to the device via fluid conduit, and the like.

The fluid delivery means may be a syringe plunger actuated manually, a syringe pump, a variable speed fluid transfer pump, a peristaltic pump, the regulated release of compressed gas, or other suitable means to supply fluids. The delivery means may also be driven manually or by mechanical means such as compression or extension springs, or other mechanical methods, by electro-mechanical means such as an electric motor, solenoid drive, or other electro-mechanical means, or by pneumatic or hydraulic means.

Over all, it is to be appreciated that terms used herein are to be interpreted and understood expansively and not strictly.

The foregoing summarizes the principal features of the invention and some of its optional aspects. The invention may be further understood by the description of the presently preferred embodiments, in conjunction with the drawings, which now follow.

### BRIEF DESCRIPTION OF THE DRAWINGS

The accompanying drawings illustrate presently preferred embodiments of the invention and, together with the description that follows, serve to explain the principles of the invention.

FIG. 1 depicts a plan and detailed view of an embodiment of the invention infusing micro bubbles within a drug carrier fluid into a patient.

FIG. 2 depicts a plan and top view of a coaxial needle embodiment of the invention with a piezo source.

FIG. 3 depicts a hollow stylet embodiment of the invention positioned within a needle.

FIG. 4 depicts a plan and detailed views of an embodiment of the invention to deliver an acoustically activated drug and a second drug in the form of micro bubbles within a carrier fluid.

FIGS. 5A and 5B depict an embodiment of the invention with a temporary storage vessel to receive and hold the bubbled fluid, for later withdrawal and injection into a patient using a needle and syringe.

### DETAILED DESCRIPTION OF PREFERRED EMBODIMENTS

Reference will now be made in detail to various suitable embodiments including a presently preferred embodiment of the invention as illustrated in the accompanying drawings. It will be understood that this description is exemplary and is to assist in understanding the invention and the principles of operation.

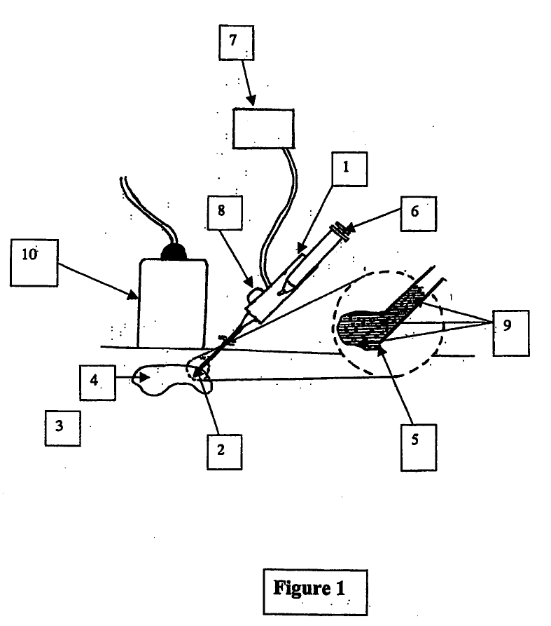

FIG. 1 depicts a plan and detailed view of the device being used to infuse a liquid drug and gas micro bubbles into a patient.

A handheld assembly (1) is used to position a needle (2) within a patient (3) at a particular point of interest (4). A liquid drug (5) in a syringe (6) is connected via fluid conduit (not depicted) in the hand held assembly (1) to the needle (2), and is delivered using the syringe plunger. A gas supply (7) with regulator (not depicted), controlled by a switch (8) on the handheld assembly, is connected to a permeable interface (not depicted) that is connected to the liquid drug fluid conduit in the hand held assembly (1). The gas supply (7) supplies the gas to be delivered through the permeable interface. As the gas passes through the permeable interface into the liquid drug fluid conduit, bubbles are generated and infused as micro bubbles (9) within the liquid drug. The liquid drug infused with micro bubbles (9) is then delivered through the needle into the patient for activation by ultrasound (10).

The gas may be compressed or pumped or driven by propellant, and may be CO₂, nitrogen, perflourochemicals, noble gases, oxygen, room air, or other types of suitable gases. It may be medical grade to lessen adverse effects on the patient.

The gas flow may be controlled and smoothed by a valve or damper system (not depicted). A fluidic valve or fluid may be connected to a permeable interface to enhance the effectiveness of the permeable interface to generate or vary the size of bubbles.

It is to be appreciated that the term “drug” as used in the specification can be liquid, a solution, a suspension, solid particulates in a solution, etc. The liquid drug may be any suitable therapeutic agent or agents that can be delivered under pressure through a needle or catheter, such as a single organic or inorganic drug, a solution of different drugs, drug particles or radiolabelled particles suspended in a fluid, a time release delivery system such as drug eluting microspheres or other embedded drug systems suspended in a fluid, an acoustically activated drug delivery system, a targeted drug delivery system or agent, or other therapeutic agents. A small quantity of the therapeutic agent, for example, 0.2 to 1.0 ml, depending on the application, may be delivered.

Acoustically activating a micro or nano bubble, either a bubble comprised of a drug or a non-therapeutic bubble within a drug carrier, with ultrasound may be used:
to activate the pharmacological activity of a therapeutic agent, such as enhancing drug transport through tissues and across cell membranes, and, or
to create a local hyperthermic condition that can enhance the destruction of diseased tissue such as cancerous tissue, and, or
to further enhance the drug uptake of acoustically activated drug systems by means such as increasing the local cavitation

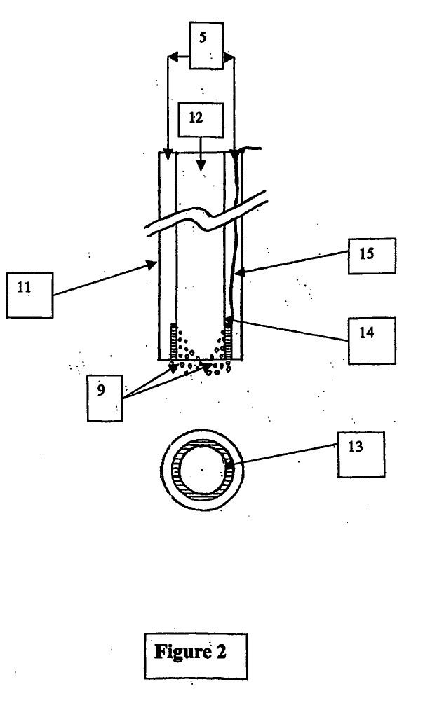

FIG. 2 depicts a plan and top view of a coaxial needle embodiment of the invention with a piezo source.

A coaxial needle (11) with an inner and outer lumen is used to deliver a liquid drug (5) and carrier fluid (12). As depicted, the liquid drug (5) is supplied to the outer lumen while the carrier fluid (12) is supplied to the inner lumen. At the proximal end of the coaxial needle (11), separate fluid conduits (not shown) connect respective inner and outer lumens of the coaxial needle (11) to a respectively liquid drug supply (not shown) and to a carrier fluid supply (not shown), such a syringes, to supply liquid drug (5) and the carrier fluid (5) into the appropriate lumens in the coaxial needle. Alternatively, liquid drug (5) may be supplied to the inner lumen and the carrier fluid (12) supplied to the outer lumen.

The needle (11) is inserted into a patient at a point of interest. The drug (5) is delivered under pressure via a fluid pump (not shown) through a permeable interface (13) to be infused into the carrier fluid flow path as micro bubbles (9). A variety of fluid pumps are suitable. The selection of a suitable pressure is dependent upon several factors, such as the size of the openings in the permeable interface, the viscosity of the fluid and the size and homogeneity of the bubbles desired. As an example, where the openings in the permeable interfaces are very small, nano scale, electro-mechanical or mechanized pumping may be necessary to generate sufficient pressure to create suitable sized bubbles.

A simple manual syringe may be used to dispense the bubbled carrier fluid for many applications. Alternatively, pressure from a fluid pump may also be used for dispensing the bubbled carrier 12 out of the coaxial needle.

A number of relatively biologically harmless fluids could serve as the bubble carrier fluid, such as sterile saline, sterile water, blood, or other fluids. The carrier fluid may include additives to alter its viscosity in order to promote the creation and stability of transient bubbles. The carrier fluid may include additives to promote the efficacy of the therapeutic agent, such as a drug to prevent infection or to aid or to combat the migration of the drug. For example, the carrier fluid may include cell membrane destabilization to reduce the cell membrane strength and improve drug uptake.

The permeable interface may be comprised of an array of tiny orifices or nozzles or a single array or orifice. The micromachining, microdrilling, or fabrication of such an interface may be accomplished by a number of established techniques on a number of different materials, such as laser machining of metals and other materials, filters of polycarbonate or other materials with orifices produced through material exposure to radiation, chemical etching, EDM machining of ultra-hard materials such as ceramics or titanium, glass or silicon processing using HF or DRIE etching, powder blasting of holes, ultrasonic drilling, fabrication of structured wafers, fluid jet drilling, and other means.

Permeable membranes, porous media, and other permeable interfaces with nano or micro sized openings may also be made from ceramic materials using a high temperature sintering process.

A piezo source (14) may be used to provide vibration. The piezo source (14) is supplied current through a cable (15) positioned within the coaxial needle (11) and connected to a switch and power source (not depicted).

The piezo source (14) depicted in FIG. 2 is the side view of an annular piezo polymer in contact with the permeable interface (13).

The piezo source may be any suitable, small transducer or transducer array, such as a piezo ceramic or a piezo polymer, such as 10 micron thick PVDF piezo polymer.

Ultrasonically vibrating the permeable interface may be to generate smaller bubbles than might be possible with a static permeable interface. Vibration may also generate a more homogenous size range of bubbles. Vibration may also be induced at less than ultrasonic frequencies through electromechanical means such as a variable speed electric motor.

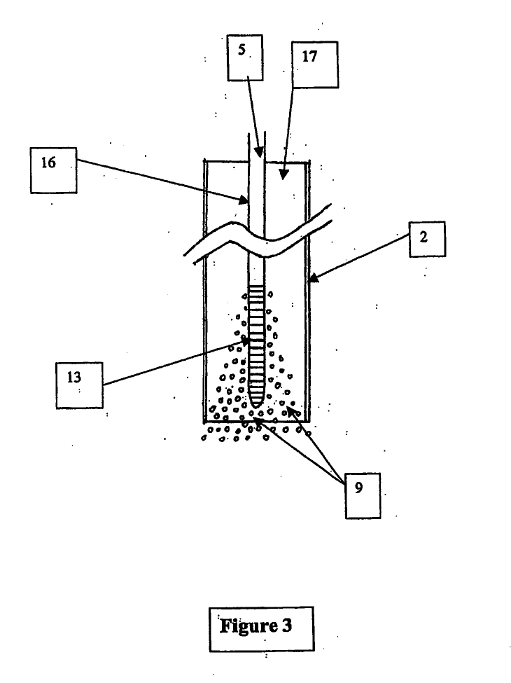

In FIG. 2 (and in FIG. 3), the permeable interface is located inside the patient at the distal tip of the needle (2) (or catheter), thereby enabling infusion into the patient and activation prior to dissipating. The permeable interface may run the length of the needle or catheter or at various positions along the length of the needle or catheter.

The coaxial needle may be fabricated with a variety of needle tip geometries including standard openings, such as spinal, chiba, franseen, and others, or a closed distal tip with slots or openings along the side of the needle, or fabricated with combinations of geometries.

FIG. 3 depicts a hollow stylet embodiment of the invention.

A hollow stylet (16) is positioned within a needle (2). A drug (5) is added to the stylet (16) and is delivered through the stylet (16) under pressure applied to the plunger of a syringe (not shown) sufficient for the drug (5) to flow through a permeable interface (13) at the distal tip. The drug is infused into a carrier fluid and drug solution (17) as microbubbles (9).

The pressure may be supplied to the drug using a variety of means including a manual syringe or an electromechanical fluid pump. The pressure required is dependent on a variety of factors, such as the size and homogenity of the bubbles desired, viscosity of the drug, and the like.

The hollow stylet may be a flexible fluid conduit positioned within a catheter (not depicted).

In this example depicted, the drug is liquid, but may be gaseous depending on the drug and the properties of the point of interest, for example, its density.

In an embodiment, not shown, the hollow stylet may be used without a needle or catheter to infuse microbubbles directly into a patient without a carrier fluid. The hollow stylet may be positioned at a desired point of interest within a patient through means such as manipulating the stylet itself, or through means such as attachment to suitable guide wires, probes, laparoscopes, endoscopes, or other surgical instruments.

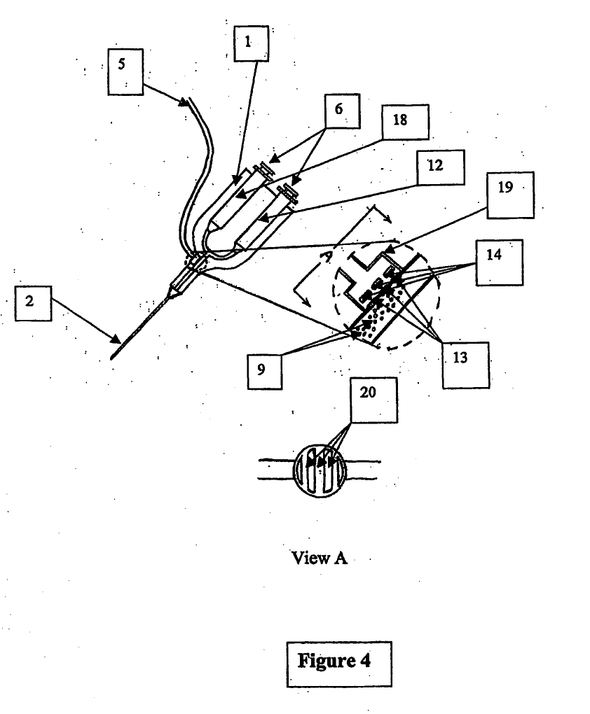

FIG. 4 depicts a plan and detailed views of an embodiment of the invention to deliver an acoustically activated, embedded drug system (18) and a liquid drug (5) in the form of micro bubbles (9) within a carrier fluid (12).

A physician uses the handheld assembly (1) to position the needle (2) at the desired point of interest (not depicted). The hand assembly comprises two syringes. A Y-shaped conduit connects the outlet of each of the syringes and provides a path for fluid flow, which then merges the two paths into a single path of fluid flow within a single body conduit. This single conduit is connected to a hollow needle that may be inserted into the point of interest, defining a path of fluid flow into the patient's body.

An acoustically activated drug (18) is contained in a syringe (6) and is delivered to the point of interest by manually depressing the plunger of the syringe (6).

A carrier fluid (12) is contained in a second syringe (6). The carrier fluid is delivered to the point of interest by manually depressing the plunger of the second syringe (6).

Liquid drug (5) is contained in a vessel and is pumped under pressure (pumping mechanism not depicted) via a flexible tubing conduit to a fluid housing (19) which connects to the single conduit on a hand held assembly (1), and then to the point of interest.

The fluid housing (19) is a solid, molded portion which defines the microfluidic flow path and provides a place onto which the permeable interface and piezo sources attach. The housing (19) is connected to the conduit through fittings. The housing (19) is designed to control the fluid flow path such that the drug flows past piezo sources (14) mounted on horizontal members (20) in the housing (19) frame and on to join into the single conduit for delivery to a point of interest. The piezo sources (14) are proximal to the permeable interfaces (13). The fluid housing may be injection molded, cast, machined or otherwise fabricated using materials such as medical grade plastics or metals.

As depicted, the syringes, conduits and fluid housing are contained in a hand held frame which includes a mount for attachment of the needle. The frame may be variously shaped containing or supporting one or more of fluid containers, conduits, and injection means. The bubble generating means may also be supported in the frame. The frame may also support various other controls, regulators, valves, heat sources refrigeration sources, temperature sensors, pressure sensors, flow sensors, fluid switch mechanisms, flow rate regulators, ultrasound transducers, transducer array, insulation, and the like. The frame may be provided with a handle. The fame may also support meters, controllers, controller I/O, display and power source. Alternatively, one or more of these components may be separate from the frame but systemically electrically linked to other components on the frame.

The drug (5) flows through flexible tubing and on through the permeable interfaces (13). Piezo sources (14) mounted on the horizontal members (20) vibrate the drug (5) at the permeable interfaces (13) in order to generate smaller, more homogenous microbubbles (9). The drug (5) is then infused into the carrier fluid (12) as microbubbles (9).

The pressure required to be applied to the liquid drug will be based on a number of factors, such as viscosity and the size/homogeneity requirements of the bubbles. Preferably, all the liquid is transformed into appropriately sized liquid bubbles, so as to avoid waste, although some of the liquid may pass into the carrier fluid in a undesired size or shape that cannot be acoustically activated.

The drug microbubbles (9) infused in the carrier fluid (12) are then delivered along the conduit following the flow path through the hollow needle (2) using the syringe (6) to be injected into the patient at the desired point of interest (not depicted)). An ultrasound source (not depicted) is used to simultaneously acoustically activate the acoustically activated drug (18) and drug microbubbles (9) at the point of interest.

Depending on the desired treatment regime, the acoustically activated drug and the drug to be infused into a carrier may be delivered to a point of interest sequentially or simultaneously as required. If the piezo source frequency is at a resonance frequency of the acoustically activated drug or in any other circumstances where avoidance of undesired activation may occur, then it may be preferable for the acoustically activated drug and the liquid drug be delivered at separate times, with the acoustically activated drug delivered prior to the generation of the bubbles with the piezo source, to avoid premature activation of the drug while in the syringe.

The handheld assembly (1) may alternatively be provided with a plurality of syringes, or other vessels containing fluids for delivery to a patient. The fluid from other vessels may be delivered under pressure using manual or mechanical means and may be connected to the single conduit.

The utility of combination therapy, the means to deliver a variety of therapeutic agents at a point of interest, is to enhance treatment efficacy for indications such as drug resistant cancer tumors. Further utility is obtained by the flexibility to vary treatment to meet a patient's specific needs.

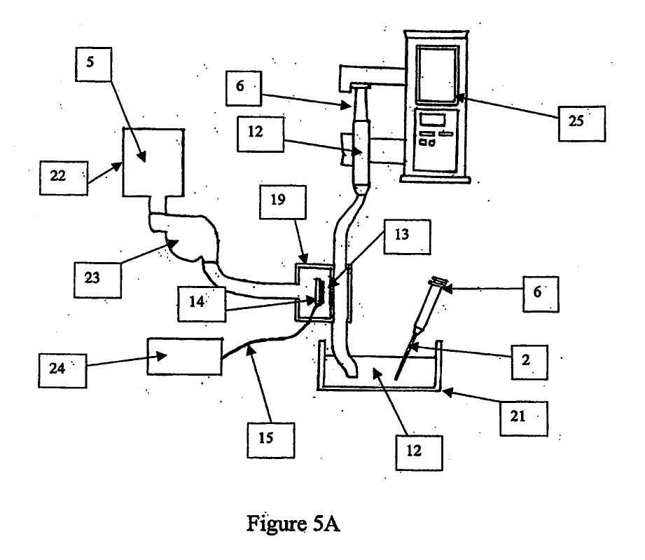

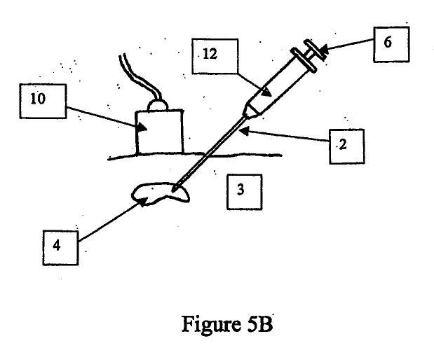

FIG. 5A depicts an embodiment of the invention with a temporary storage vessel (21) to receive and hold the bubble fluid, for later withdrawal and injection into a patient (3) using a needle (2) and syringe (6).

A liquid drug (5) in a vessel (22) is delivered under pressure with a variable speed fluid transfer pump (23) connected to a housing (19). The housing (19) is a solid, molded part that defines the microfluidic flow path of the liquid drug (5) to the permeable interface (13), and onto which the permeable interface (13) and piezo sources (14) are mounted. A power source (24) delivers current through a cable (15) to the piezo sources (14). The piezo sources (14) vibrate the liquid drug (5) as it is delivered under pressure through the permeable interface (13) to be infused in microbubble form (not depicted) in the carrier fluid (12).

The carrier fluid (12) is contained within a syringe (6) mounted to a programmable, DC motor driven, commercial infusion pump (25). The infusion pump (25), such as a Baxter AS50, is used to control the delivery of the carrier fluid (12) such that the concentration of microbubbles (not depicted) per unit volume of carrier fluid is controlled. The infusion pump (25) delivers the carrier fluid (12) infused with microbubbles into a temporary storage vessel (21).

A needle (2) and syringe (6) is used to withdraw the carrier fluid (12) infused with microbubbles from the storage vessel (21).

FIG. 5B depicts the syringe (6) being used to deliver the carrier fluid (12) infused with micro bubbles through a needle (2) and into a patient (3) to a particular point of interest (4) such as a solid tumor. An ultrasound source (10) is then used to activate the microbubbles to enhance the therapeutic efficacy of the treatment.

The various embodiments of the device may be comprised of or used with standard, commercial, medical components such as needles, needle adaptors, catheters, syringes, guide wires, infusion pumps, fluid conduits, leak proof fittings, meters, laparoscopes, endoscopes, probes, multiple lumen delivery means and the like. The various embodiments of the device may be comprised of specialized components with attributes such as MRI compatible materials, coatings to enhance the image guidance of the device, and the like. Fluid vessels, such as syringes, may be attached to the handheld assembly using means such as adjustable clamps or connected to the handheld assembly using means such as fluid conduits.

A medical device such as disclosed in PCT/CA2004/000174 which is incorporated herein by reference may be provided with an additional means to generate micro or nano scale bubbles. For example, FIG. 3A of PCT/CA2004/000174 may be provided with a permeable membrane positioned within the flexible fluid conduit into which therapeutic fluid flows. The therapeutic fluid flows through the permeable interface under pressure, and is infused into the echogenic fluid, thereby generating micro or nano scale bubbles for delivery to a point of interest. These micro or nano scale bubbles may be used to enhance the ultrasonic visibility of a needle as disclosed in PCT/CA2004/000174 and may also be used to permit therapy enhanced by acoustic activation as disclosed in this application. Alternatively, there may be provided a third syringe containing a carrier fluid which may be connected to the flexible conduit downstream of the permeable membrane. The therapeutic fluid would be infused into the carrier fluid, generating micro or nano bubbles.

### CONCLUSION

Devices for generating transient bubbles for infusion within a patient and for activation by an ultrasound source are disclosed. The devices may enhance the efficacy of a treatment by increasing cellular uptake of a drug at the point of interest and may reduce undesired side effects.

The transient bubbles may be comprised of a therapeutic agent delivered in a carrier fluid, or may be generated within a therapeutic agent that acts as the carrier fluid, or may be generated within a carrier fluid to be infused in combination with a therapeutic agent. The bubbles may be generated from a therapeutic agent to be delivered directly into a patient without a carrier fluid.

The device may be comprised of a handheld assembly or system comprising injection means for injecting fluids into a patient such as a needle or catheter, fluid containers, fluid discharge means, and a bubble generating means to generate micro or nano scale bubbles.

The device may be further comprised of a piezo source for vibrating the permeable interface and/or the fluid in proximity to the permeable interface.

The device may include means for controlling or regulating the fluid supply, such as flow controls, pressure sensors, flow sensors, fluid switch mechanisms, regulators or valves. The device may include meters, controllers, controller I/O, display, and power source.

The device may have a variety of applications, for example, be used to enhance the treatment of liver tumours by ethanol injection.

These claims, and the language used therein, are to be understood in terms of the variants of the invention, which have been described. They are not to be restricted to such variants, but are to be read as covering the full scope of the invention as is implicit within the invention and the disclosure that has been provided herein.

The foregoing has constituted a description of specific embodiments showing how the invention may be applied and put into use. These embodiments are only exemplary. The invention in broader, and more specific aspects, is further described and defined in the claims that now follow.

1-15. (canceled)

16. A medical device for locally generating transient bubbles for introduction into a body comprising:
a fluid container for a fluid;
a flow path communicating with the fluid container for directing fluid flow from said fluid container, said flow path being defined at least in part by the fluid container;
a fluid discharge means for applying a pressure to the fluid causing the fluid to travel the flow path,
a bubble generating means positioned in the flow path for generating transient bubbles comprised of the fluid, and
optionally, an injection means communicating with the flow path for directing and conveying the fluid travelling the flow path into a body at a point of interest.

17. The device according to claim 16 further comprising:
a second container for a second fluid;
a second flow path communicating with the second fluid container for directing fluid flow from said second fluid container, said second flow path being defined at least in part by the second container; and
a second fluid discharge means for applying a pressure to the second fluid causing the second fluid to travel the second flow path,
wherein the second flow path joins the first flow path, at least in part, downstream of the bubble generating means.

18. The device according to claim 16, wherein the fluid and/or second fluid comprises one or more of a therapeutic agent, a carrier, an additive, a stabilizer or a surfactant, a plurality of therapeutic agents, carriers, additives, or surfactants, and/or a combination thereof.

19. The device according to claim 18, wherein the therapeutic agent includes one or more of:
a. a liquid drug,
b. a solid drug suspended in a fluid,
c. an acoustically activated drug delivery system, suspended in a fluid,
d. a radioisotope labelled drug or particle,
e. an imaging system contrast agent for imaging systems including CT scans, MRI, ultrasound, or X-ray.

20. The device according to claim 16, wherein all or part of the flow path and/or second flow path is defined in a needle, a catheter, a stylet, a guide wire, a laparoscope, an endoscope, or a multiple lumen needle, or a multiple lumen catheter.

21. The device according to claim 16, wherein the bubble generating means comprises one or more of a permeable interface, a nano or micro scale valve, or a nano or micro scale fluid conduit.

22. The device according to claim 16, further comprising vibration means, preferably a piezo source, for vibrating the bubble generating means or the fluid proximate to the vibrating means, and, optionally, wherein the piezo source frequency or power output is adjustable in real time.

23. The device according to claim 16, wherein at least a part of the device is adapted to be hand held for directing fluid traveling the flow path to the body at the point of interest.

24. The device according to claim 16, further comprising temperature control means for modulating a temperature of the fluid and/or second fluid, said temperature control means preferably including one or more of a refrigeration source, a heating source, a temperature sensor, a temperature control, or a thermal insulating material.

25. The device according to claim 16, wherein a flow rate of the fluid is adjustable in real-time.

26. The device according to claim 16, wherein the fluid container is a syringe and the fluid discharge means is a plunger slidably disposed within the syringe.

27. The device of according to claim 16, further comprising a storage vessel positioned downstream of the bubble generating means for receiving the first fluid, wherein the pressure causes the first fluid to travel the flow path into the temporary storage vessel.

28. A medical device for generating transient bubbles comprising:
a plurality of fluid containers, each having a fluid discharge means disposed in connection with each container for applying a pressure on a fluid in the container,
a plurality of fluid conduits, each connected to the container at a first end and each connected to a body conduit at a second end,
an injection means connected to the body conduit for discharging the fluid from a distal end, the injection means adapted to convey the fluid into a body at a point of interest,
a bubble generating means disposed in at least one of the plurality of fluid conduits,
wherein for each container, a flow path is defined from the container through the fluid conduit, the body conduit and the injection means for discharge from the distal end, wherein for each container a pressure from the fluid discharge means causes the fluid to travel the flow path, and wherein a first fluid in the one of the plurality of fluid conduits travels the flow path through bubble generating means for generating transient bubbles comprised of the first fluid.

29. The device according to claim 28, wherein the fluid and/or second fluid comprises one or more of a therapeutic agent, a carrier, an additive, a stabilizer or a surfactant, a plurality of therapeutic agents, carriers, additives, or surfactants, and/or a combination thereof.

30. The device according to claim 29, wherein the therapeutic agent includes one or more of:
a. a liquid drug,
b. a solid drug suspended in a fluid,
c. an acoustically activated drug delivery system, suspended in a fluid,
d. a radioisotope labelled drug or particle,
e. an imaging system contrast agent for imaging systems including CT scans, MRI, ultrasound, or X-ray.

31. The device according to claim 28, wherein all or part of the flow path and/or second flow path is defined in a needle, a catheter, a stylet, a guide wire, a laparoscope, an endoscope, or a multiple lumen needle, or a multiple lumen catheter.

32. The device according to claim 28, wherein the bubble generating means comprises one or more of a permeable interface, a nano or micro scale valve, or a nano or micro scale fluid conduit.

33. The device according to claim 28, further comprising vibration means, preferably a piezo source, for vibrating the bubble generating means or the fluid proximate to the vibrating means, and, optionally, wherein the piezo source frequency or power output is adjustable in real time.

34. The device according to claim 28, wherein at least a part of the device is adapted to be hand held for directing fluid traveling the flow path to the body at the point of interest.

35. The device according to claim 28, further comprising temperature control means for modulating a temperature of the fluid and/or second fluid, said temperature control means preferably including one or more of a refrigeration source, a heating source, a temperature sensor, a temperature control, or a thermal insulating material.

36. The device according to claim 28, wherein a flow rate of the fluid is adjustable in real-time.

37. The device according to claim 28, wherein the fluid container is a syringe and the fluid discharge means is a plunger slidably disposed within the syringe.

38. The device of according to claim 28, further comprising a storage vessel positioned downstream of the bubble generating means for receiving the first fluid, wherein the pressure causes the first fluid to travel the flow path into the temporary storage vessel.

39. A system for acoustically activating the bubbles comprising:
a. a device according to claim 16; and
b. acoustic activation means for generating acoustic pulses,
wherein the acoustic activation means is preferably selected from the group consisting of an extracorporeal ultrasound transducer, a transducer array for transmitting pulses, or an internal ultrasound transducer or transducer array for transmitting pulses, and more preferably, further comprising means for focusing the pulses such as a time reversal acoustics array.

40. A system for acoustically activating the bubbles comprising:
a. a device according to claim 28; and
b. acoustic activation means for generating acoustic pulses,
wherein the acoustic activation means is preferably selected from the group consisting of an extracorporeal ultrasound transducer, a transducer array for transmitting pulses, or an internal ultrasound transducer or transducer array for transmitting pulses, and more preferably, further comprising means for focusing the pulses such as a time reversal acoustics array.

41. A method for generating transient bubbles in a fluid comprising:
providing a fluid,
applying a pressure to the fluid causing the fluid to travel a flow path,
passing the fluid through a bubble generating means positioned in the flow path for generating transient bubbles comprised of the fluid,
optionally, combining the fluid with a second fluid in the flow path,
optionally, injecting the fluid into a body; and
optionally further comprising transmitting an ultrasonic pulse at the bubbles.
